(19) United States
(12) Patent Application Publication
(10) Pub. No.: US 2026/0033707 A1
(43) Pub. Date: Feb. 5, 2026

(54) GAS/WATER VALVE WITH BACKFLOW PREVENTION FOR MEDICAL DEVICES

(71) Applicant: Boston Scientific Scimed, Inc., Maple Grove, MN (US)

(72) Inventors: Jennifer Evelyn Edmondson, Marlborough, MA (US); Brian Luis, Boylston, MA (US); Kate Beatrice Concannon, Brighton, MA (US)

(73) Assignee: Boston Scientific Scimed, Inc., Maple Grove, MN (US)

(21) Appl. No.: 19/288,314

(22) Filed: Aug. 1, 2025

Related U.S. Application Data
(60) Provisional application No. 63/679,237, filed on Aug. 5, 2024.

(51) Int. Cl. A61B 1/015; F16K 11/07

(52) U.S. Cl. CPC A61B 1/015; F16K 11/0716

(57) ABSTRACT

A gas/water valve for a medical device, including a shaft extending from a proximal end to a distal end and having a bore extending from a first opening to a vent (e.g., a second opening) proximate the proximal end. A seal assembly may be positioned around the shaft. The seal assembly may comprise a distal sealing member including at least one gas flow aperture extending through a thickness thereof and a proximal sealing member. The proximal sealing member may be axially displaceable along the shaft.

### CROSS-REFERENCE TO RELATED APPLICATIONS

This application claims the benefit of U.S. Provisional Patent Application Ser. No. 63/679,237 filed on Aug. 5, 2024, the disclosure of which is incorporated herein by reference.

### FIELD

This disclosure relates generally to medical devices, and particularly to a gas/water valve for use with an endoscope.

### BACKGROUND

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. A control section of the endoscope may include a suction cylinder and a gas/water cylinder. Valves may be inserted into these cylinders to control various functions of the endoscope. For example, a gas/water valve for an endoscope may be inserted into the gas/water cylinder or channel of the endoscope to provide air and water to the endoscope. When the gas/water valve is in a neutral position, air may escape from a vent in the valve. When insufflation is desired, an operator may place a finger over the vent, which redirects the air towards the distal end of the endoscope thus insufflating the working lumen. When the operator engages the gas/water valve (e.g., by depressing or otherwise actuating the valve), air is redirected to a fluid container and creates pressure in the container that may cause water (or other fluid) to flow towards the distal end of the endoscope.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a gas/water valve for a medical device may include a shaft extending from a proximal end to a distal end and having a bore extending from a first opening to a second opening proximate the proximal end, a seal assembly positioned around the shaft. The seal assembly may include a distal sealing member including at least one gas flow aperture extending through a thickness thereof and a proximal sealing member. The proximal sealing member may be axially displaceable along the shaft.

Alternatively or additionally to any of the examples above, in another example, the distal sealing member may comprise an annular body having an outer diameter.

Alternatively or additionally to any of the examples above, in another example, the at least one gas flow aperture may be positioned between a central bore and an outer perimeter of the distal sealing member.

Alternatively or additionally to any of the examples above, in another example, the proximal sealing member may comprise an annular body having an outer diameter.

Alternatively or additionally to any of the examples above, in another example, the outer diameter of the proximal sealing member may be less than the outer diameter of the distal sealing member.

Alternatively or additionally to any of the examples above, in another example, the outer diameter of the proximal sealing member may be approximately equal to the outer diameter of the distal sealing member.

Alternatively or additionally to any of the examples above, in another example, the proximal sealing member may be configured to selectively seal the at least one gas flow aperture.

Alternatively or additionally to any of the examples above, in another example, the proximal sealing member may include at least one raised region.

Alternatively or additionally to any of the examples above, in another example, the at least one raised region may be configured to mate with the at least one gas flow aperture.

Alternatively or additionally to any of the examples above, in another example, when the gas/water valve is in a neutral position, the proximal sealing member may be configured to seal the at least one gas flow aperture of the distal sealing member.

Alternatively or additionally to any of the examples above, in another example, when the gas/water valve is in a first position different from the neutral position, the proximal sealing member may be configured to be proximally displaced from the distal sealing member.

Alternatively or additionally to any of the examples above, in another example, when the gas/water valve is in a second position different from the neutral position, the proximal sealing member may be configured to seal the at least one gas flow aperture of the distal sealing member.

Alternatively or additionally to any of the examples above, in another example, the gas/water valve may further comprise at least one connection member extending between the distal sealing member and the proximal sealing member.

Alternatively or additionally to any of the examples above, in another example, the at least one gas flow aperture may comprise two or more gas flow apertures.

Alternatively or additionally to any of the examples above, in another example, the distal sealing member may be more rigid than the proximal sealing member.

In another example, a gas/water valve for a medical device may comprise a shaft extending from a proximal end to a distal end and having a bore extending from a first opening to a vent proximate the proximal end, wherein the shaft has at least one groove and at least one ridge, a button cap coupled to the shaft proximate the proximal end of the shaft, a resilient member coupled to the button cap, and a seal assembly positioned within the at least one groove. The seal assembly may comprise a distal sealing member including at least one gas flow aperture extending through a thickness thereof and a proximal sealing member, the proximal sealing member axially displaceable along the shaft. In a first configuration, the proximal sealing member may be configured to selectively block a flow of gas through the at least one gas flow aperture and in a second configuration the proximal sealing member may be proximally spaced from the at least one gas flow aperture to allow a flow of gas therethrough.

Alternatively or additionally to any of the examples above, in another example, the at least one gas flow aperture may comprise\ a first generally “C” shaped aperture and a second generally “C” shaped aperture.

Alternatively or additionally to any of the examples above, in another example, in the first configuration a distal side of the proximal sealing member may contact a proximal side of the distal sealing member.

Alternatively or additionally to any of the examples above, in another example, the gas/water valve may further comprise at least one elastic connection member extending between the distal sealing member and the proximal sealing member.

In another example, a gas/water valve for a medical device may comprise a shaft extending from a proximal end to a distal end and having a bore extending from a first opening to a vent proximate the proximal end, wherein the shaft has at least one groove and at least one ridge, a button cap coupled to the shaft proximate the proximal end of the shaft, a resilient member coupled to the button cap, and a seal assembly positioned within the at least one groove. The seal assembly may comprise an annular distal sealing member fixedly secured to the shaft, the annular distal sealing member including two or more gas flow apertures extending through a thickness thereof, the two or more gas flow apertures positioned between a central aperture and an outer perimeter of the annular distal sealing member and an annular proximal sealing member, the proximal sealing member axially displaceable along the shaft. In a first configuration, the proximal sealing member may be configured to selectively block a flow of gas through the two or more gas flow apertures and in a second configuration the proximal sealing member may be proximally spaced from the two or more gas flow apertures to allow a flow of gas therethrough.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various example configurations and together with the description serve to explain the principles of the present disclosure.

FIG. 1 schematically depicts components of an illustrative configuration of an endoscope;

FIG. 2 schematically depicts components of an illustrative configuration of an endoscope system;

FIG. 3 schematically depicts a perspective view of an illustrative configuration of a gas/water valve;

FIG. 4A schematically depicts a cross-sectional view of the illustrative configuration of the gas/water valve of FIG. 3 in a valve well with the gas/water valve in a neutral position;

FIG. 4B schematically depicts a cross-sectional view of the illustrative configuration of the gas/water valve of FIG. 3 in a valve well with the gas/water valve in a first position;

FIG. 4C schematically depicts a cross-sectional view of the illustrative configuration of the gas/water valve of FIG. 3 in a valve well with the gas/water valve in a second position;

FIG. 5A is a schematic perspective view of an illustrative configuration of a seal assembly in a first configuration;

FIG. 5B is a schematic perspective view of the illustrative configuration of the seal assembly of FIG. 5A in a second configuration;

FIG. 6 is a schematic perspective view of the illustrative configuration of the seal assembly of FIG. 5A including optional connection members; and

FIG. 7 is a schematic perspective view of an illustrative configuration of a seal assembly in a first configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. Additionally, terms that indicate the geometric shape of a component/surface refer to exact and approximate shapes.

Embodiments of the present disclosure are described with specific reference to a gas/water valve of an endoscope. Such embodiments may be used to control the flow of fluid, for a variety of different purposes where it is desired to selectively fluidly couple one or more channels of a device.

Although the present disclosure includes descriptions of a gas/water valve suitable for use with an endoscope system to control insufflation and/or lens wash or irrigation fluid to an endoscope, the devices, systems, and methods herein could be implemented in other medical systems requiring the selective fluid and/or gas coupling of various channels, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation, and lens wash as a means of flushing debris, cleaning optics, and insufflating the working lumen. Gas/water valves may be used for operating gas and/or fluid flow.

Gas/water valves may incorporate a one-way seal to prevent backflow and maintain insufflation pressure in the working lumen. In some cases, one-way seals may partially or fully invert during use. This may increase the amount of force required to depress the valve and/or remove the valve. Additionally, a partially or fully inverted seal may reduce the effectiveness of the seal when insufflation is required. It may be desirable to provide gas/water valves with alternative sealing mechanisms.

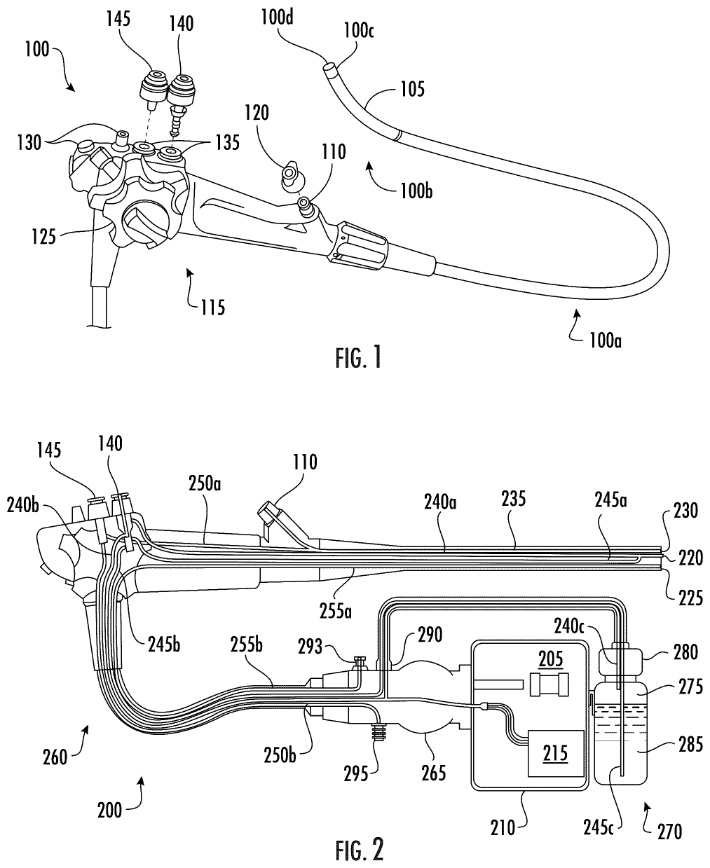

With reference to FIGS. 1 and 2, an illustrative endoscope 100 and endoscope system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of a gas/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash supply tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash supply tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an illustrative operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash supply tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash supply tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash supply tubing 245c, may be placed in the path of the irrigation supply tubing to help prevent back-flow into the reservoir after water has passed the valve. In some cases, irrigation water may be supplied from the water reservoir 270. Some illustrative systems where the supply tubing for irrigation and lens wash are connected to and drawn from a single water reservoir are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference.

When the suction valve 145 is in a neutral position or rest state, without the user's finger on and/or depressing the valve, air is allowed to flow into and/or out of the valve 145 to atmosphere. When suction is desired, the user's finger is used to block a vent to atmosphere as well as to depress the suction valve. Depressing the suction valve 145 may move the suction valve 145 within the valve well 135 to fluidly couple the working channel 235 with the suction pump (not explicitly shown) via the suction supply line 250a.

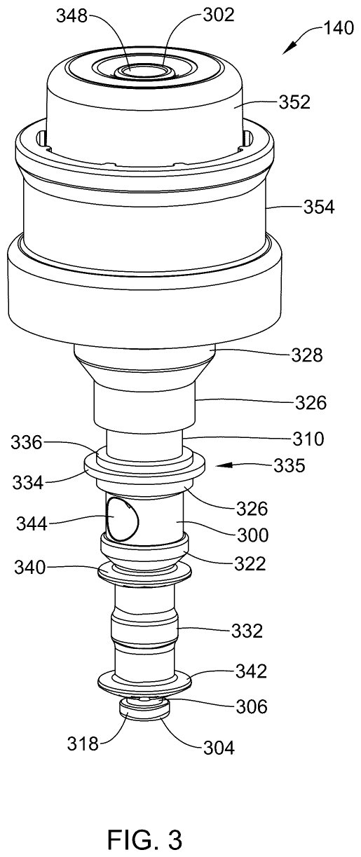

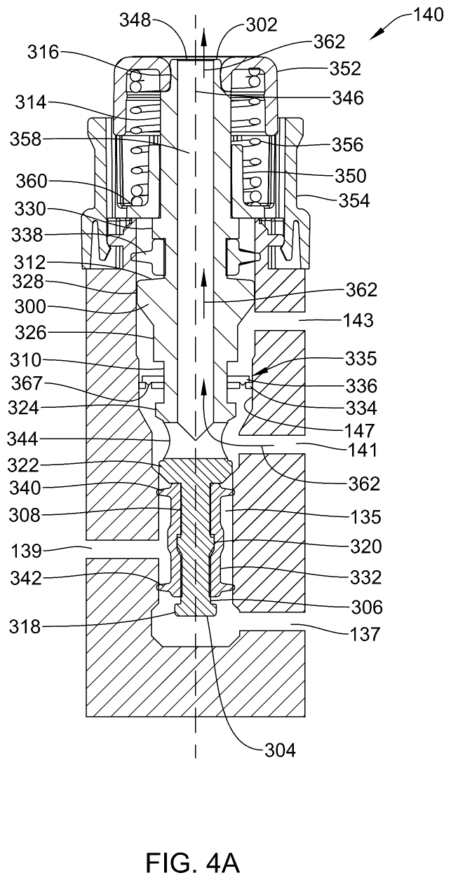

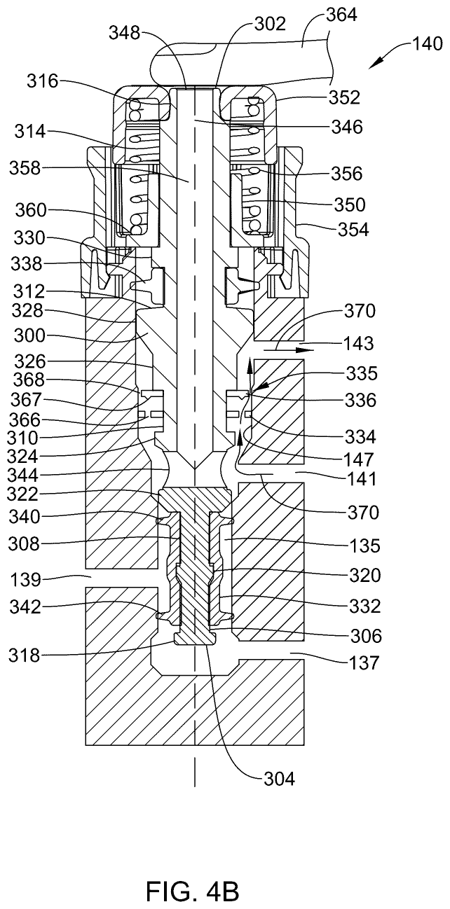

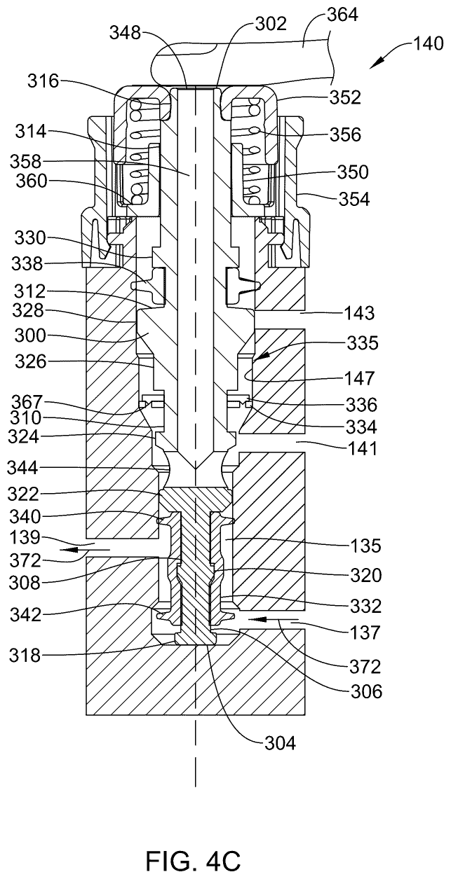

FIG. 3 is a schematic perspective view of an illustrative configuration of the gas/water valve 140 that may be used with the endoscope 100. FIG. 4A is a schematic cross-sectional view of the gas/water valve 140 disposed within a valve well 135 with the gas/water valve 140 in a neutral position. FIG. 4B is a schematic cross-sectional view of the gas/water valve 140 disposed within a valve well 135 with the gas/water valve 140 in a first position. FIG. 4C is a schematic cross-sectional view of the gas/water valve 140 disposed within a valve well 135 with the gas/water valve 140 in a second position.

Referring to FIGS. 3 and 4A-4C, the gas/water valve 140 may have a stem or shaft 300 extending from a first or proximal end 302 to a second or distal end 304. The shaft 300 may include a plurality of grooves 306, 308, 310, 312, 314, 316 and a plurality of ridges 318, 320, 322, 324, 326, 328, 330 that are molded and/or machined as part of the shaft 300. Said differently, the outer diameter or cross-sectional dimension of the shaft 300 may intermittently increase and/or decrease along the length thereof from the first end 302 to the second end 304. In some cases, the changes in cross-sectional dimension may occur in an abrupt or stair-step manner. In other examples, the changes in the cross-sectional dimension may be gradual, tapered, sloped, or non-existent. The gas/water valve 140 may further include a plurality of seals 332, 334, 336, 338 positioned along a length of the shaft 300. In addition, gas/water valve 140 may also include an inner ring 350, a button cap 352, an outer ring 354, and a biasing member 356, such as, but not limited to, a spring, rubber, elastic, or the like.

Generally, the seals 332, 334, 336, 338 may be configured to selectively fluidly isolate or fluidly couple portions of the gas/water valve 140 and/or the valve well 135. For example, depending on the position of the gas/water valve 140 relative to the valve well 135 and/or a position of the user's finger 364, the seals 332, 334, 336, 338 may be positioned to selectively fluidly couple fluid paths of the endoscope 100 and endoscope system 200. As will be described in more detail herein, the seals 332, 334, 336, 338 may prevent gas/air from entering the water channel (e.g., water inlet 137 and/or water outlet 139) and prevent gas/air from exiting anywhere other than outside of a vent hole 348 on the top of the shaft 300 or through a gas outlet 143 in the valve well 135.

In some embodiments, one or more of the plurality of seals 332, 334, 336, 338 can be fixedly or permanently attached to the shaft 105, such as, for example, by over-molding. In other embodiments, one or more of the plurality of seals 332, 334, 336, 338 can be removably attached to or coupled to the shaft 300, such as for example, by sliding the seal onto shaft 300.

The seals 332, 334, 336, 338 may be formed from any material suitable for sealing a portion of the shaft 300. For example, the plurality of seals 332, 334, 336, 338 can be formed from polyurethane, polyurea, polyether (amide), polyether block amide (PEBA), thermoplastic elastomeric olefin, copolyester, and styrenic thermoplastic elastomer, carbon fiber, glass fiber, ceramics, methacrylates, poly (N-isopropylacrylamide), poly(ethylene oxide)-poly(propylene oxide)-poly(ethylene oxide) (PEO-PPO-PEO) co-polymers, rubber, plastic (e.g., polycarbonates), acrylonitrile butadiene styrene (ABS), methylmethacrylate acrylonitrile butadiene styrene (MABS), silicone, etc., or combinations thereof.

A first seal 332 may be positioned in a first groove 306 and may include a first sealing region 340 positioned proximal to a water outlet 139 of the valve well 135 and a second sealing region 342 positioned distal to the water outlet 139. The first and second scaling regions 340, 342 may contact a surface of the valve well 135 to provide a seal between the gas/water valve 140 and the valve well 135. In the neutral or first position (FIGS. 4A and 4B, respectively), the second sealing region 342 may be positioned proximal to a water inlet 137 of the valve well 135 and while in the second position (FIG. 4C), the shaft 300 of the gas/water valve 140 may be distally displaced to position the second sealing region 342 distal to or generally aligned with the water inlet 137. When the gas/water valve 140 is in the neutral position or the first position, the second sealing region 342 fluidly isolates the water outlet 139 from the water inlet 137. Further, in the neutral position, the first position, or the second position, the first sealing region 340 fluidly isolates the water inlet 137 and the water outlet 139 from the air/gas inlet 141 and the air/gas outlet 143. In some cases, a diameter of the valve well 135 may increase proximate the water inlet 137 to allow the water inlet 137 to be fluidly coupled with the cavity of the valve well 135 and the water outlet 139 without requiring part or all of the second sealing region 342 to be positioned distal to the water inlet 137.

A distal sealing member 334 (e.g., a second seal) and a proximal scaling member 336 (e.g., a third seal) may form a seal assembly 335. The seal assembly 335 may be positioned within another groove 310 proximal to the first seal 332 and proximal to an opening 344 in shaft 300. The distal seal member 334 and/or the proximal seal member 336 may contact a surface 147 of the valve well 135 to provide a seal between the gas/water valve 140 and the valve well 135. In some examples, the distal scaling member 334 may be fixedly secured to the shaft 300 while the proximal sealing member 336 may be configured to be axially displaced along a longitudinal axis 346 of the gas/water valve 140. The distal sealing member 334 may be over-molded with the shaft 300. However, this is not required. The distal sealing member 334 may be secured to the shaft 300 using other fixation techniques, such as, but not limited to, adhesives, plastic welding, thermal bonding, friction fits, or the like. As will be described in more detail herein, the distal and proximal sealing members 334, 336 may be configured to selectively fluidly couple the gas/air inlet 141 of the valve well 135 with the gas/air outlet 143 of the valve well 135.

A fourth seal 338 may be positioned within another groove 312 proximal to the gas/air inlet and outlet 141, 143. The fourth seal 338 may contact the inner surface of the valve well 135 to provide a seal to fluidly isolate the gas/air inlet and outlet 141, 143 from atmosphere external to the gas/water valve 140.

The opening 344 of the shaft 300 may be fluidly coupled with a bore or lumen 358 extending proximally from the opening 344 (e.g., a first opening) to the vent opening 348 (e.g., a second opening). The bore 358 may extend colinearly with the longitudinal axis 346 of the gas/water valve 140 and/or the opening 344 may extend generally orthogonal to the longitudinal axis 346 of the gas/water valve 140. However, other suitable configurations are contemplated. The opening 344 may extend at non-orthogonal angles relative to the longitudinal axis of the gas/water valve 140, as desired. In some embodiments, the opening 344 may extend from a first side to a second opposing side of the shaft 300. Said differently, the opening 344 may extend through a thickness of the shaft 300. When the vent opening 348 is not covered by an operator, gas/air may enter the gas/water valve 140 via the opening 344, travel through the bore 358, and exit the gas/water valve 140 via the vent opening 348, as shown at arrows 362.

The inner ring 350 may have a diaphragm or collar 360 extending from an outer circumference of the inner ring 350 towards the internal circumference of the outer ring 354. The resilient member 356 may be installed to be positioned between the inner ring 350 and the button cap 352 such that one end of the resilient member 356 is secured to the diaphragm or collar 360 and the other end to the button cap 352. In some embodiments, the inner ring 350 is a single monolithic internally molded part of the outer ring 354 while in other embodiments these may be two separate components. In various embodiments, the outer ring 354 (along with the inner ring 350 when molded as a monolithic part of the outer ring 354) is molded using material of sufficient rigidity. The inner ring 350, outer ring 354, and the button cap 352 respectively define internal bores for receiving the proximal end 302 or vent 348 of the shaft 300. The proximal end 302 of the shaft 300 may be placed through the inner ring 350 and the resilient member 356 and secured to the button cap 352. When assembled, the diaphragm or collar 360 of the inner ring 350 may rest upon a ridge 330.

Referring to FIG. 4A, in the neutral position, the vent 348 is uncovered or unblocked. Gas/air may enter the valve well 135 via the gas/air inlet 141. Gas enters the gas/water valve 140 via the opening 344, travels through the bore 358, and exits the gas/water valve 140 via the vent 348, as shown at arrows 362. The distal and proximal scaling members 334, 336 provide a gas-tight seal between the shaft 300 of the gas/water valve 140 and the inner wall 147 of the valve well 135 between the gas/air inlet 141 and the gas/air outlet 143 to prevent gas/air from entering the gas/air outlet 143 when the gas/water valve 140 is in the neutral position.

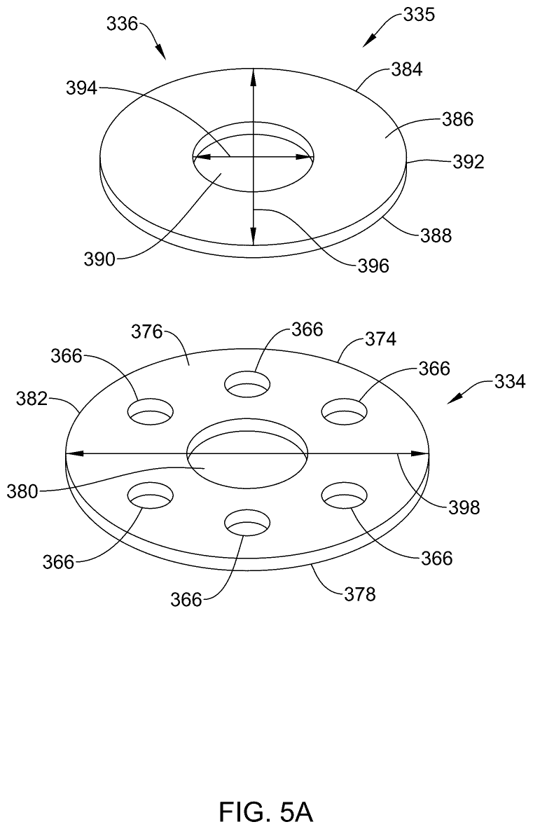

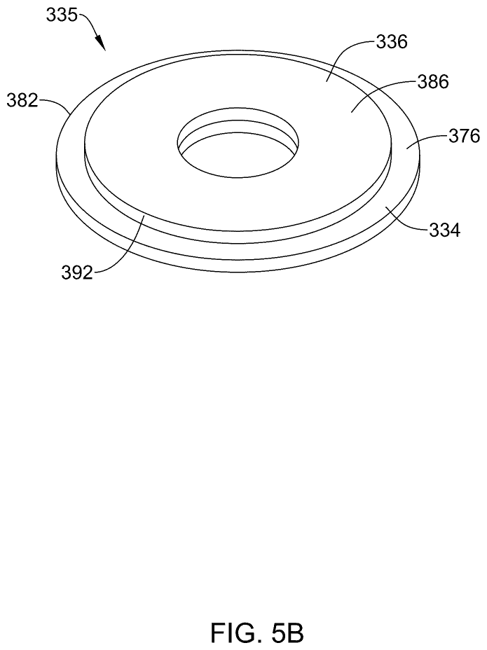

Referring to FIG. 4B, in the first position, the user covers or blocks the vent 348, using, for example, but not limited to, a finger 364. When the vent 348 is blocked, gas/air that flows into the valve well 135 via the gas/air inlet 141 is prevented from exiting the gas/water valve 140 via the vent 348. The gas/air accumulates in the valve well 135 and the bore 358 of the shaft 300 and the pressure within the valve well 135 increases. The distal sealing member 334 may include one or more openings or apertures 366 extending through a thickness of the distal sealing member 334. The pressure generated by the accumulating gas/air may pass through the apertures 366 and push against a distal surface of the proximal sealing member 336. The proximal sealing member 336 may be movably secured relative to the shaft 300 such that the pressure or air flow through the apertures 366 proximally displaces the proximal sealing member 336. The proximal scaling member 336 may move proximally along the shaft 300 (e.g., within the groove 310) until the proximal sealing member 336 abuts or contacts a distal end of the ridge 326. However, this is not required. In some cases, the proximal movement of the proximal sealing member 336 may be limited by the flow rate of gas/air through the apertures 366. In some embodiments, the proximal sealing member 336 may have an outer diameter 396 (see, for example, FIG. 5A) that is less than a diameter of the valve well 135 proximate the seal assembly 335, such that when the proximal sealing member 336 is axially displaced towards the proximal end of the groove 310, there is a gap or opening 368 between an outer edge of the proximal sealing member 336 and the wall 147 of the valve well 135. The gap 368 may be an annular opening extending around a circumference of the wall 147 of the valve well 135 and/or around the outer circumference of the proximal sealing member 336. In some embodiments, the gap 368 may extend around an entirety of the circumference of the wall 147 of the valve well 135 and/or an entirety of the outer circumference of the proximal sealing member 336. In other embodiments, the gap 368 may extend around less than an entirety of the circumference of the wall 147 of the valve well 135 and/or less than an entirety of the outer circumference of the proximal sealing member 336. The gap 368 may provide a fluid path for gas/air to flow from the gas/air inlet 141 through the valve well 135 and out the gas/air outlet 143 to provide insufflation, as shown at arrows 370.

In other embodiments, the diameter of the valve well 135 may increase from a first diameter proximate the distal end of the groove 310 to a second diameter proximate the proximal end of the groove 310. The second diameter may be greater than the first diameter. The transition between the first diameter may be gradual, tapered, or sloped or may be an abrupt stair-step transition in diameter. The outer diameter of the distal and proximal sealing members 334, 336 may be approximately equal to or greater than the first diameter of the valve well 135 to provide a gas/air-tight seal when the distal and proximal sealing members 334, 336 are positioned proximate the distal end of the groove 310. The outer diameter of the proximal sealing member 336 may be less than the second diameter of the valve well 135 such that when the proximal sealing member 336 is axially displaced towards the proximal end of the groove 310, there is a gap or opening between an outer edge of the proximal sealing member 336 and the wall of the valve well 135.

Referring now to FIG. 4C, when the gas/water valve 140 is in the second position, the gas/water valve 140 is axially displaced in the distal direction while the vent 348 remains blocked or covered. In the second position, the first seal 332 is axially displaced in the distal direction to fluidly couple the water inlet 137 and the outer water outlet 139. Water flows into the valve well 135 via the water inlet 137, fills the annular cavity surrounding the first seal 332, and exits the valve well 135 via the water outlet 139, as shown at arrows 372. As the gas/water valve 140 is distally displaced, the distal and proximal sealing members 334, 336 are also distally displaced. Further, the proximal sealing member 336 may be distally displaced to bring the proximal sealing member 336 into contact with the distal sealing member 334 to seal the apertures 366 thereof. This may cause pressurization of the water container 270 to provide pressurized water to the distal end of the endoscope. In some embodiments, when the gas/water valve 140 is in the second position, the proximal sealing member 336 may be proximally spaced from the distal sealing member 334 and the proximal sealing member 336 may provide a gas-tight seal between the gas/air inlet 141 and the gas/air outlet 143. In other embodiments, the distal side of the proximal sealing member 336 may contact the proximal side of the distal sealing member 334 when the gas/water valve 140 is in the second position.

FIG. 5A is a perspective view of the distal sealing member 334 and the proximal sealing member 336 in an axially spaced configuration. As described above, the distal sealing member 334 and the proximal sealing member 336 are axially spaced when the gas/water valve 140 is in the first position (e.g., FIG. 4B) and may sometimes be axially spaced when the gas/water valve 140 is in the second position (e.g., FIG. 4C). The distal sealing member 334 may have a generally annular body 374 having a thickness extending from a proximal side 376 to a distal side 378. A central aperture 380 may extend through the thickness of the body 374. As described above, the distal sealing member 334 may be over-molded with the shaft 300 such that the shaft 300 extends through the central aperture 380. It is contemplated that the distal sealing member 334 is fixedly coupled with the shaft 300 such that the distal sealing member 334 is not axially displaced along the longitudinal axis 346 of the shaft 300. The distal sealing member 334 may further include one or more gas/air flow apertures 366 extending through the thickness thereof. The one or more gas/air flow apertures 366 may be positioned between the central apertures 380 and an outer edge or perimeter 382 of the distal sealing member 334. The outer perimeter 382 may extend between the proximal side 376 and the distal side 378.

The distal sealing member 334 may include any number of gas/air flow apertures 366 desired, such as, but not limited to, one, two, three, four, five, six, or more. In some embodiments, the gas/air flow apertures 366 may be formed by injection molding the distal sealing member 334. In other embodiments, the gas/air flow apertures 366 may be formed by drilling or otherwise cutting the apertures 366 from an annular disc.

The gas/air flow apertures 366 may be uniformly arranged or spaced circumferentially around the body 374. For example, in an embodiment which includes six gas/air flow apertures 366, the gas/air flow apertures 366 may be spaced approximately 60° from one another. However, this is not required. In other embodiments, the gas/air flow apertures 366 may be eccentrically arranged, as desired.

A radial positioning of the gas/air flow apertures 366 may vary, as desired. In some cases, the gas/air flow apertures 366 may be radially spaced from the central aperture 380 by a same distance. In other examples, the gas/air flow apertures 366 may be radially spaced from the central aperture 380 by differing distances. For example, some of the gas/air flow apertures 366 may be spaced a first distance from the central aperture 380 while other gas/air flow apertures 366 may be spaced a second distance from the central aperture 380. It is contemplated that the gas/air flow apertures 366 may be arranged in any configuration desired. However, the gas/air flow apertures 366 may be spaced a maximum distance from the central aperture 380 such that the proximal sealing member 336 may fully cover the gas/air flow apertures 366 when the gas/water valve 140 is in the neutral position, as will be described in more detail herein.

The gas/air flow apertures 366 may have a generally circular cross-sectional shape. However, this is not required. The gas/air flow apertures 366 may have any cross-sectional shape desired, such as, but not limited to, square, rectangular, oval, polygonal, triangular, eccentric, or the like.

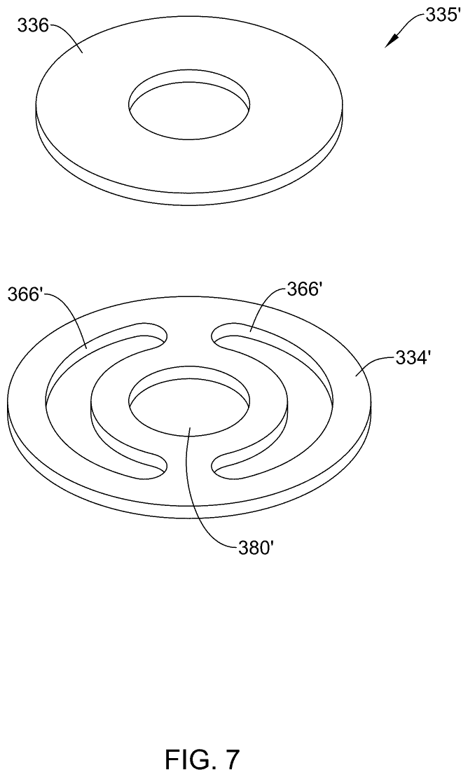

Referring briefly to FIG. 7 which illustrates a perspective view of the seal assembly 335 including a distal sealing member 334′ having alternatively shaped gas/air flow apertures 366′, in some examples, the gas/air flow apertures 366′ may be sized and/or shaped to increase the flow rate of air traveling through the gas/air flow apertures 366′ while still allowing for sealing of the gas/air flow apertures 366′. In FIG. 7, the distal sealing member 334′ may include two gas/air flow apertures 366′ each having a generally curved bean or “C” shape radially spaced from a central aperture 380′. The gas/air flow apertures 366′ may be separated by regions of the distal sealing member 334′ free from apertures 366′. In some cases, the gas/air flow aperture 366′ may be a single annular aperture or a single aperture that is less than a complete annular ring. In addition to increasing a flow rate of the gas/air, it is contemplated that reducing the number of gas/air flow apertures 366′ may simplify the manufacturing process for injection molding and/or reduce the overall amount of material required for injection molding.

Returning to FIG. 5A, in some embodiments, the outer perimeter 382 of the distal sealing member 334 may be contoured to provide better sealing contact with the wall of the valve well 135. In some cases, the outer perimeter 382 may include a sloped edge. For example, the outer diameter of the distal sealing member 334 may increase from the distal side 378 to the proximal side 376. The reverse configuration is also contemplated in which the outer diameter of the distal sealing member 334 decreases from the distal side 378 to the proximal side 376. In other examples, the outer perimeter 382 of the distal sealing member 334 may have a curved or convex shape. It is contemplated that the outer perimeter 382 of the distal sealing member 334 may take other shapes, as desired.

The proximal sealing member 336 may have a generally solid annular body 384 having a thickness extending from a proximal side 386 to a distal side 388. A central aperture 390 may extend through the thickness of the body 384. An outer perimeter 392 may extend between the proximal side 386 and the distal side 388. The proximal scaling member 336 may be axially movable relative to the shaft 300 with the shaft 300 extending through the central aperture 390. In some embodiments, a diameter 394 of the central aperture 390 may be greater than an outer diameter of the shaft 300 proximate the groove 310 such that an entirety of the proximal sealing member 336 may be axially displaced relative to the shaft 300. For example, the diameter 394 of the central aperture 394 may be large enough to allow the proximal sealing member 336 to move proximally and distally along the shaft 300 while being tight enough to ensure leakage isn't occurring. However, this is not required. In some cases, the diameter 394 of the central aperture 390 may be the same as the outer diameter of the shaft 300 proximate the groove 310. In some instances, an entirety of the proximal sealing member 336 may not be axially displaced. For example, in some examples, the radially inwards portion of the proximal sealing member 336 may not be axially displaced or may be axially displaced to a lesser extent than a radially outwards portion of the proximal scaling member 336.

In some embodiments, the outer perimeter 392 of the proximal scaling member 336 may be contoured to provide better sealing contact with the wall of the valve well 135 in configurations where the proximal sealing member is designed to contact the wall of the valve well 135. In some cases, the outer perimeter 392 may include a sloped edge. For example, the outer diameter of the proximal sealing member 336 may increase from the distal side 388 to the proximal side 386. The reverse configuration is also contemplated in which the outer diameter of the proximal sealing member 336 decreases from the distal side 388 to the proximal side 386. In other examples, the outer perimeter 392 of the proximal sealing member 336 may have a curved or convex shape. It is contemplated that the outer perimeter 392 of the proximal sealing member 336 may take other shapes, as desired.

FIG. 5B is a perspective view of the distal sealing member 334 and the proximal sealing member 336 when the gas/water valve 140 is in the neutral position. The distal side 388 of the proximal sealing member 336 is positioned against and contacting the proximal side 376 of the distal sealing member 334. The proximal scaling member 336 may be formed from a material that is more flexible than the material used to form the distal sealing member 334. This may allow the proximal sealing member 336 to seal the gas/air flow apertures 366 in a gas-tight manner. For example, the distal side 388 of the proximal sealing member 336 may fully cover the gas/flow apertures 366 to prevent a flow of gas/air therethrough when the gas/water valve 140 is in a neutral position. In some embodiments, the distal side 388 of the proximal sealing member 336 may fully cover the gas/air flow apertures 366 to prevent a flow of gas/air therethrough when the gas/water valve 140 is in the second position to prevent backflow of gas/air when insufflation is not active. Said differently, when insufflation is no longer required, the user may remove their finger (or other blocking mechanism) from the vent 348. Pressure within the gas/air outlet 143 may move the proximal seal 336 in a distal direction as the pressure distal to the distal seal 334 is relieved (via the vent) to cover the gas/air flow apertures 366 preventing backflow of gas/air.

The distal side 388 of the proximal sealing member 336 may be contoured to mate with the gas/air flow apertures 366 of the distal sealing member 334. For example, the distal side 388 of the proximal sealing member 336 may include optional raised regions 367 (see, for example, FIGS. 4A-4C), such as, but not limited to, convex or domed regions configured to mate with and seal with the gas/air flow apertures 366 of the distal sealing member 334. The raised regions 367 may or may not increase the thickness of the proximal sealing member 336 relative to regions of the proximal sealing member 336 free from the raised regions 367. The raised regions 367 may be sized and shaped to mate with corresponding apertures 366 in the distal sealing member 334. In the illustrated example, the proximal sealing member 336 may include six raised regions 367 with each raised region 367 mating with a corresponding gas/air flow aperture 366 of the distal sealing member 334.

It is contemplated that in some embodiments, an outer diameter 396 of the proximal seal 336 may be less than an outer diameter 398 of the distal seal 334. This may increase the area gas/air may flow through when gas/water valve 140 is in the first position to provide insufflation. It is contemplated that the outer diameter 396 of the proximal seal 336 may extend radially beyond the one or more gas/air flow apertures 366 such that the proximal seal 336 fully covers the one or more gas/air flow aperture to prevent backflow when insufflation is not desired. In other embodiments, the outer diameter 396 of the proximal sealing member 336 may be the same as or approximately the same as the outer diameter 398 of the distal sealing member 334.

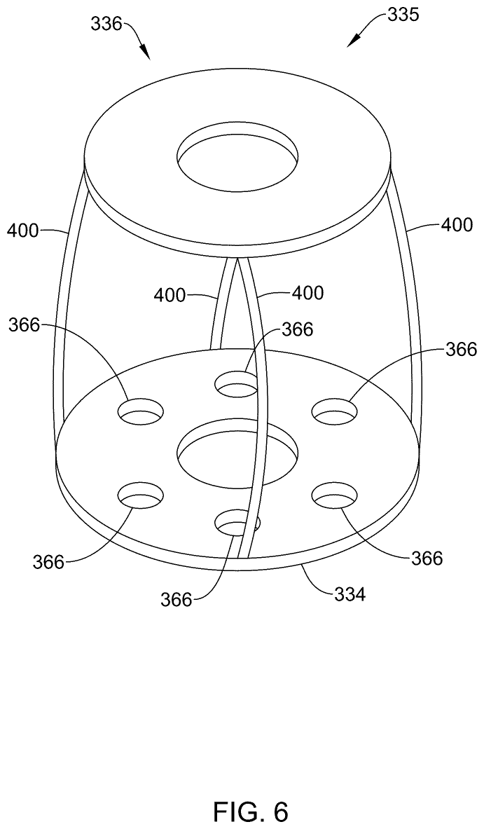

FIG. 6 is a perspective view of the illustrative seal assembly 335 including one or more optional connection members 400 extending between the distal sealing member 334 and the proximal sealing member 336. The connection members 400 may be configured to limit a distance the proximal sealing member 336 moves from the distal sealing member 334 when the gas/water valve 140 is in the first position. It is contemplated that the connection members 400 may decrease the amount of time required to seal the gas/air flow apertures 366 after insufflation to prevent backflow and/or leakage. In some cases, the connection members 400 may be formed from an elastic material that stretches to allow the proximal sealing member 336 to move proximally when the gas/water valve 140 is in the first position and contracts to returns to an original length to pull the proximal sealing member 336 distally when insufflation is complete (e.g., the gas/water valve 140 is used in the neutral or second positions) or when pressure in the patient builds up causing gas/air flow in a proximal direction. The seal assembly 335 may include any number of connection members 400 desired, such as, but not limited to, one, two, three, four, or more. The connection members 400 may be uniformly arranged or spaced circumferentially around the outer perimeters 382, 392 of the distal and proximal sealing members 334, 336. For example, in an embodiment which includes four connection members 400, the connection members 400 may be spaced approximately 90° from one another. However, this is not required. In other embodiments, the connection members 400 may be eccentrically arranged, as desired. It is further contemplated that the connection members 400 may be secured to other radial locations on the distal and/or proximal sealing members 334, 336 in addition to or in place of the outer perimeters 382, 392 thereof.

In some examples, the connection members 400 may have a ribbon-like shape. For example, the connection members 400 may have a thickness that is less than a width thereof. Said differently, the connection members 400 may have a generally rectangular cross-sectional shape. However, this is not required. The connection members 400 may have other cross-sectional shapes, such as, but not limited to, square, circular, oblong, polygonal, triangular, eccentric, or the like. In other examples, the connection members 400 may be wires, threads, or filaments. In yet other examples, the connection members 400 may each include two or more twisted, woven, or braided filaments.

It is contemplated that the length of the connection members 400 may be selected to moderate a flow rate of gas/air during insufflation. For example, decreasing a length of the connection members 400 may limit the proximal displacement of the proximal scaling member 336 and may allow for less gas/air flow. Increasing the length of the connection members 400 may allow for a greater distance of travel of the proximal sealing member 334 and may increase the gas/air flow during insufflation.

When the seal assembly 335 includes one or more connection members 400, the distal sealing member 334 and the proximal sealing member 336 may be positioned over the shaft 300 after manufacture of the shaft 300. However, this is not required. In some cases, the distal sealing member 334, the one or more connection members 400, and the proximal sealing member 336 may be over-molded onto the shaft 300. In such an instance, the proximal sealing member 336 may include a connection to the shaft 300 that can be broken after over-molding to allow the proximal sealing member 336 to be axially displaced. In another example, the distal sealing member 334 and/or proximal scaling member 336 may be molded or 3-D printed with the connection members 400. A dissolvable or removable support material may extend between the distal sealing member 334 and/or proximal sealing member 336 and/or the connection members 400. In yet another example, the distal sealing member 334 and the proximal sealing member 336 may be molded separately. The connection members 400 may then be secured to the distal sealing member 334 and the proximal sealing member 336. This assembly may then be assembled onto the shaft 300.

Various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as illustrative only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A gas/water valve for a medical device, the gas/water valve comprising:
a shaft extending from a proximal end to a distal end and having a bore extending from a first opening to a second opening proximate the proximal end;
a seal assembly positioned around the shaft, the seal assembly comprising:
a distal sealing member including at least one gas flow aperture extending through a thickness thereof; and
a proximal sealing member, the proximal sealing member axially displaceable along the shaft.

2. The gas/water valve of claim 1, wherein the distal sealing member comprises an annular body having an outer diameter.

3. The gas/water valve of claim 2, wherein the at least one gas flow aperture is positioned between a central bore and an outer perimeter of the distal sealing member.

4. The gas/water valve of claim 1, wherein the proximal sealing member comprises an annular body having an outer diameter.

5. The gas/water valve of claim 4, wherein the outer diameter of the proximal sealing member is less than the outer diameter of the distal sealing member.

6. The gas/water valve of claim 4, wherein the outer diameter of the proximal sealing member is approximately equal to the outer diameter of the distal sealing member.

7. The gas/water valve of claim 1, wherein the proximal sealing member is configured to selectively seal the at least one gas flow aperture.

8. The gas/water valve of claim 1, wherein the proximal sealing member includes at least one raised region.

9. The gas/water valve of claim 8, wherein the at least one raised region is configured to mate with the at least one gas flow aperture.

10. The gas/water valve of claim 1, wherein when the gas/water valve is in a neutral position, the proximal sealing member is configured to seal the at least one gas flow aperture of the distal sealing member.

11. The gas/water valve of claim 10, wherein when the gas/water valve is in a first position different from the neutral position, the proximal sealing member is configured to be proximally displaced from the distal sealing member.

12. The gas/water valve of claim 10, wherein when the gas/water valve is in a second position different from the neutral position, the proximal sealing member is configured to seal the at least one gas flow aperture of the distal sealing member.

13. The gas/water valve of claim 1, further comprising at least one connection member extending between the distal sealing member and the proximal sealing member.

14. The gas/water valve of claim 1, wherein the at least one gas flow aperture comprises two or more gas flow apertures.

15. The gas/water valve of claim 1, wherein the distal sealing member is more rigid than the proximal sealing member.

16. A gas/water valve for a medical device, the gas/water valve comprising:
a shaft extending from a proximal end to a distal end and having a bore extending from a first opening to a vent proximate the proximal end, wherein the shaft has at least one groove and at least one ridge;
a button cap coupled to the shaft proximate the proximal end of the shaft;
a resilient member coupled to the button cap; and
a seal assembly positioned within the at least one groove, the seal assembly comprising:
a distal sealing member including at least one gas flow aperture extending through a thickness thereof; and
a proximal sealing member, the proximal sealing member axially displaceable along the shaft;
wherein in a first configuration, the proximal sealing member is configured to selectively block a flow of gas through the at least one gas flow aperture and in a second configuration the proximal sealing member is proximally spaced from the at least one gas flow aperture to allow a flow of gas therethrough.

17. The gas/water valve of claim 16, wherein the at least one gas flow aperture comprises a first generally “C” shaped aperture and a second generally “C” shaped aperture.

18. The gas/water valve of claim 16, wherein in the first configuration a distal side of the proximal sealing member contacts a proximal side of the distal sealing member.

19. The gas/water valve of claim 16, further comprising at least one elastic connection member extending between the distal sealing member and the proximal sealing member.

20. A gas/water valve for a medical device, the gas/water valve comprising:
a shaft extending from a proximal end to a distal end and having a bore extending from a first opening to a vent proximate the proximal end, wherein the shaft has at least one groove and at least one ridge;
a button cap coupled to the shaft proximate the proximal end of the shaft;
a resilient member coupled to the button cap;
a seal assembly positioned within the at least one groove, the seal assembly comprising:
an annular distal sealing member fixedly secured to the shaft, the annular distal sealing member including two or more gas flow apertures extending through a thickness thereof, the two or more gas flow apertures positioned between a central aperture and an outer perimeter of the annular distal sealing member; and
an annular proximal sealing member, the proximal sealing member axially displaceable along the shaft;
wherein in a first configuration, the proximal sealing member is configured to selectively block a flow of gas through the two or more gas flow apertures and in a second configuration the proximal sealing member is proximally spaced from the two or more gas flow apertures to allow a flow of gas therethrough.
